(19) United States
(12) Patent Application Publication
(10) Pub. No.: US 2018/0140616 A1
(43) Pub. Date: May 24, 2018

(54) METHODS OF TREATMENT OF, AND FORMULATIONS FOR TREATMENT OF ANORECTAL AND GENITOURINARY CONDITIONS

(71) Applicant: William Jow, Aberdeen, NJ (US)

(72) Inventor: William Jow, Aberdeen, NJ (US)

(21) Appl. No.: 15/876,101

(22) Filed: Jan. 19, 2018

Related U.S. Application Data
(63) Continuation-in-part of application No. 12/927,845, filed on Nov. 26, 2010.

(51) Int. Cl. A61K 31/573; A61K 31/58; A61K 31/56; A61K 31/165; A61K 31/137; A61K 31/553; A61K 31/506; A61K 31/55; A61K 31/343; A61K 31/4458; A61K 31/439; A61K 31/38; A61K 31/335; A61K 31/381; A61K 31/15; A61K 31/42; A61K 31/53; A61K 31/5375; A61K 31/496; A61K 31/4525; A61K 31/435; A61K 31/19; A61K 47/44; A61K 47/10; A61K 47/12; A61P 29/00; A61P 1/00; A61P 31/04; A61P 31/12; A61P 31/00

(52) U.S. Cl. CPC A61K 31/573; A61P 31/00; A61K 31/56; A61K 31/165; A61K 31/137; A61K 31/553; A61K 31/506; A61K 31/55; A61K 31/343; A61K 31/4458; A61K 31/439; A61K 31/38; A61K 31/335; A61K 31/381; A61K 31/15; A61K 31/42; A61K 31/53; A61K 31/5375; A61K 31/496; A61K 31/4525; A61K 31/435; A61K 31/19; A61K 47/44; A61K 47/10; A61K 47/12; A61P 29/00; A61P 1/00; A61P 31/04; A61P 31/12; A61K 31/58

(57) ABSTRACT

This invention relates to a novel method of treating various inflammation-related anorectal and genitourinary conditions using various related, novel formulations. The formulations comprise, at a minimum, a corticosteroid, an antidepressant agent(s) with anti-allergic and/or anti-itch properties and a cooling/soothing agent(s). Selection of specific ingredients depends on the nature of the target tissues, the etiologies of inflammation and the severity of symptoms. To maximize its efficacy, each of the formulations has a unique combination of several active ingredients, and each of these ingredients exerts its anti-inflammatory effect at various phases of the inflammatory cascade, resulting in a synergistic effect of pain relief and healing. To minimize the side effects, the current multi-active ingredient formulations allow each of these ingredients to exist at a lower concentration than the existing single-active ingredient formulations, thus minimizing the likelihood for side effects due to systemic absorption and toxicity. The formulations are highly versatile in that the composition of the ingredients can be varied and adjusted to treat a wide array of anorectal and genitourinary conditions.

### CROSS-REFERENCE TO RELATED APPLICATIONS

This application is a continuation-in-part of U.S. patent application Ser. No. 12/927,845, filed Nov. 26, 2010, entitled “Methods of treatment of, and formulations for treatment of inflammation-related medical conditions,” and claims priority to U.S. provisional patent application Ser. No. 61/283,665, filed Dec. 7, 2009, the disclosures of which are incorporated by reference herein in their entireties.

### BACKGROUND

### Field of the Invention

This invention relates to a novel method of treating various inflammation-related medical conditions comprising anorectal, genitourinary, musculoskeletal and dermatological conditions using various related, novel formulations. Consumers and caregivers are often frustrated over.

### SUMMARY

Embodiments of the present disclosure generally relate to a novel method of treating various inflammation-related medical conditions comprising anorectal, genitourinary, musculoskeletal and dermatological conditions using various related, novel formulations. In exemplary embodiments, a topically-administered composition for treating inflammation-related medical conditions may include active ingredients and inactive ingredients, the active ingredients that may include (a) a corticosteroid ingredient, or salt thereof, selected from the group consisting of alclometasone dipropionate, amcinonide, beclomethasone, betamethasone benzoate, betamethasone dipropionate, betamethasone propionate, betamethasone valerate, budesonide, clobetasol propionate, clobetasone butyrate, clocortolone pivolate, cortisone, desonide, desoximetasone, dexamethasone, diflorasone diacetate, flumethasone pivolate, flunisolide, fluocinolone acetonide, fluocinonide, flurandrenolide, fluticasone propionate, halobetasol propionate, halocinonide, hydrocortisone, hydrocortisone acetate, hydrocortisone butyrate, hydrocortisone probutate, hydrocortisone propionate, hydrocortisone valerate, methylprednisolone, mometasone furoate, prednicarbate, prednisolone, prednisone acetate, prednisone valerate, triamcinolone acetonide, and triamcinolone hexaceto.

### DETAILED DESCRIPTION

This invention relates to a novel method of treating various inflammation-related medical conditions including anorectal and genitourinary conditions using various related, novel formulations. Consumers and caregivers are often frustrated over the relative lack of effective topical medical products in treating certain inflammatory medical conditions. Frequently, even after the acute infections or local traumas have subsided, with or without the use of anti-microbial agents, the inflammatory cascade lingers on, thus leading to chronic or recurrent inflammatory conditions. Symptoms attributable to such local inflammatory cascades may not respond to systematic treatments, which are often costly and morbid in the long run. Conversely, existing topical formulations are often limited by their suboptimal efficacy and slow relief of symptoms. This invention is a method of utilizing various combinations of topical preparations aiming at providing rapid relief and healing of acute, chronic and recurrent inflammatory anorectal and genitourinary conditions while minimizing the side effects of each of the individual ingredients. The formulations are highly versatile in that the composition of the ingredients can be varied and adjusted to treat a wide array of inflammatory conditions in these organ systems.

Each of the formulations has a unique combination of several active ingredients, and each of these ingredients exerts its anti-inflammatory effects at various phases of the inflammatory cascade resulting in a synergistic effect of pain relief and healing. The first major active ingredient in the current formulations is selected from a member of the low-, mid-, or high-potency corticosteroids, which are known for their potent anti-inflammatory effects against a wide range of inflammations. Its anti-inflammatory mechanism is mediated through inhibition of prostaglandin synthesis and leukocyte response. Topical corticosteroid preparations, when applied in small quantities, have the advantage of modulating local immune response at the target sites while avoiding systemic side effects and drug interactions. The second major active ingredient described herein is an antidepressant agent(s) with anti-allergic and/or anti-itch properties which can counteract the propagation of the inflammatory cascade through anti-histaminic, serotonin and/or neuronal pathways. The third major active ingredient, a cooling/soothing agent(s), is crucial in providing immediate relaxation and soothing of the inflamed tissues such as the epithelial, sub-epithelial, mucosal tissues and nerve endings, and in aiding tissue penetration of other active ingredients, thus resulting in an instant relief of symptoms at the target sites. All these ingredients are combined in a commercially accessible cream base, with the overall efficacy resulting from the synergistic effect achieved via combining all or some of the above ingredients are superior to that of the individual ingredient alone.

The antidepressant agents described herein specifically comprise antidepressants with anti-allergic and/or anti-itch properties, which can counteract the propagation of the inflammatory cascade through anti-histaminic, serotonin and/or neuronal pathways. They are intended to synergistically augment the anti-inflammatory potency of the corticosteroids and the anti-histaminic agents. Certain oral antidepressants have been implicated by some researchers as being capable of providing anti-allergic relief, presumably through anti-histaminic, serotonin and/or neuronal pathways (Hoffer, A., Journal of Orthomolecular Psychiatry, 1980, 9(3):164-170, http://www.orthomed.org/resources/papers/hofdepr.htm).

For dermatological applications, the use of oral antidepressants following topical corticosteroid administration in treating lichen simplex chronicus has been found to be superior to conventional oral anti-histamines (Sanjana, V. D. & Fernandez, R. J., Indian J. Dermatol. Venereol. Leprol., 1992, 58:384-387). It follows that antidepressants, when added to other anti-inflammatory ingredients in various formulations of topical preparations as described herein, may enhance the overall safety margins while lessening the overall sedative side effects, thus achieving superior anti-inflammatory, anti-allergic and anti-itch effects.

U.S. Pat. No. 6,890,544 to McCadden discloses a gel composition for the topical treatment of poison ivy and other forms of contact dermatitis. The formulations included a corticosteroid, two drying agents consisting of calamine and zinc oxide, water and an anti-itch agent(s) selected from the group consisting of menthol, camphor, phenol, benzocaine, diphenylhydramine and pramoxine. The current invention differs substantially from the above cited patent in several ways including the fact that this invention contains a major active ingredient comprising an antidepressant with anti-allergic and/or anti-itch properties.

For anorectal and genitourinary applications, while there is an abundance of formulations comprising corticosteroids and/or pramoxine, the use of a cooling agent(s) for treating such conditions for such organ systems is a rare application. For example, a non-prescriptive, mentholated herbal cream received good testimonials for multi-organ usages, including anorectal and genitourinary applications (Shaklee's Green Cream: www.barefoot-sun.com/2006/06/whats-in-your-medicine-cabinet.html).

The addition of an anti-depressant with anti-allergic and/or anti-itch properties and a cooling or soothing agent(s) to a corticosteroid and an emollient/moisturizing agent(s) for the treatment of anorectal and genitourinary conditions is unknown in the prior art. By way of example, the addition of a cooling agent(s) to a topical analgesic was demonstrated to facilitate the penetration of the latter, thus enhancing its efficacy, by effecting changes in the epidermal ultra structure (Liu, Y., et al, International J. Pharmaceutics, 2005, 305(1-2):31-36).

In another study, a cooling agent(s), when added to a cetylated fatty acid topical cream, was shown to reduce pain and improve the functional performance in individuals with arthritis (Kraemer, W., et al, J. Strength and Conditioning Research, 2005, 19(2):475-480).

In summary, while there is an abundance of patents and literature on the various combinations of active ingredients in treating various inflammation-related medical conditions, the addition of an anti-depressant with anti-allergic and/or anti-itch properties described herein to the other two major active ingredients comprising a corticosteroid and a cooling or soothing agent(s) in treating various anorectal and genitourinary conditions has not been described in the prior art.

The invention relates to novel methods of treatment of various inflammation-related anorectal and genitourinary conditions using various related, novel formulations containing the selected active ingredients, which are dispersed in commercially accessible base formulations such as a cream. The invention also relates to novel methods of treatment of various anorectal and genitourinary conditions using various novel formulations containing the three major ingredients as described above. Specific compositions of the three major active ingredients are selected depending on the nature of the target tissues, the etiologies of inflammation and the severity of symptoms.

Taken altogether, the multi-active ingredient formulations described herein allow each of these ingredients to exist at a lower concentration than existing single-active ingredient formulations, thus minimizing the likelihood for side effects due to systemic absorption and toxicity. The latter has been the major concern for oral or parenteral administrations of pharmaceutical agents. Such symptoms, in the case of prolonged corticosteroid use, may include reversible hypothalamic-pituitary-adrenal axis suppression, manifestations of Cushing's syndrome, hyperglycemia and glucosuria, and in the case of excessive application of an anti-histaminic/anti-allergic, anti-itch agent(s), may include drowsiness and sedation. On the contrary, systemic absorption leading to clinical symptoms is a rare occurrence using the formulations described for anorectal and genitourinary administrations wherein only a small amount of the formulated product is applied, for example, on the tip of a finger, pad, swab, cloth or an applicator, and then transferred to the target sites. Alternatively, formulations may be delivered via a patch, providing immediate or controlled release of the formulation.

The formulations of this invention contain lower amounts of each of the multiple active ingredients in lieu of a formulation that contains a larger concentration of a single active ingredient. This is because in the latter preparation, the single active ingredient will have to exist in a much larger quantity in order to achieve the comparable clinical efficacy as the multi-active ingredient formulations. For example, the concern for local untoward side effects such as atrophic changes, telangiectasia and depigmentation of the skin, the mucosal/epithelial linings, the anal verge and vaginal introitus following long-term use of topical steroids at a high concentration can be effectively lessened. The idea leading to the current formulation development parallels the concept of utilizing the multi-drug approach in developing modern chemotherapeutic regimens, i.e., delivering a higher overall efficacy with a wider safety margin and fewer complications when compared to the single-drug regimens.

This invention relates to novel methods of treatment of various inflammation-related anorectal and genitourinary conditions using various novel formulations containing the three major ingredients as described above comprising a corticosteroid, an antidepressant agent(s) with anti-allergic and/or anti-itch properties and a cooling/soothing agent(s). As further described, the above selected active ingredients are dispersed in commercially accessible cream base formulations. Specific ingredients are selected depending on the nature of the target tissues, the etiologies of inflammation and the severity of symptoms.

The invention consists of a method of utilizing various combinations of topical preparations aiming at providing rapid relief and healing of acute, chronic and recurrent inflammatory anorectal and genitourinary conditions while minimizing the side effects of each of the individual ingredients. The formulations are highly versatile in that the composition of the ingredients can be varied and adjusted to treat a wide array of inflammatory conditions in these organ systems.

To maximize its efficacy, each of the formulations has a unique combination of several active ingredients, and each of these ingredients exerts its anti-inflammatory effect at various phases of the inflammatory cascade, resulting in a synergistic effect of pain relief and healing.

To minimize the side effects, the current multi-active ingredient formulations allow each of these ingredients to exist at a lower concentration than the existing single-active ingredient formulations, thus minimizing the likelihood for side effects due to systemic absorption and toxicity.

Over the years as a practicing urologist, I have observed how some patients suffered from untoward systemic side effects of various hormonally based vaginal creams/gels/lotions, etc., including gynecomastia, flushing, skin irritations, vulvar ulcerations, etc. These topical preparations are often administered in large quantities (e.g., with a vaginal syringe applicator) in order to elicit adequate clinical response. However, such a conventional administration of a high quantity of a single drug locally can often cause significant local symptoms and even irreversible skin changes. Similar observations were made on my patients who were given long-term, full-strength corticosteroid creams/gels/lotions, etc. for various dermatological conditions, and subsequently developed severe atrophic dermatitis, skin thinning, hypopigmentation, easy bruisibility, skin cracking and bleeding, etc.

The various components in specific combinations can raise the thresholds for toxicities and side effects of each of their individual components, while achieving a superior outcome due to their synergistic effects, e.g., relieving the inflammatory dermatological conditions while avoiding skin thinning and other side effects associated with long-term corticosteroid use.

In order to maximize efficacy, the claimed compositions have unique combinations of several active ingredients, and each of these ingredients exerts its anti-inflammatory effect at various phases of the inflammatory cascade, resulting in a synergistic effect of pain relief and healing.

In order to minimize side effects, the current claimed multi-active ingredient compositions allow each of these ingredients to exist at a lower concentration than the existing single-active ingredient compositions, thus minimizing the likelihood for side effects due to systemic absorption and toxicity.

A method of treatment of and formulations for treatment of anorectal conditions.

The pathophysiology of symptoms related to various anorectal conditions shares the final common inflammatory pathway of pain and swelling of the tissues involved, such as the anal verge, perineum, hemorrhoidal tissues/veins, anal ulcerations, fissure and fistula formation and anal, peri-anal, rectal and peri-rectal mucosal and epithelial linings, leading to venous congestion/thrombosis, mucosal and submucosal edema/inflammation, etc. All treatment options aim at relief of tissue swelling and inflammation, the leading cause of pain and discomfort. Current topical preparations for treatments of anorectal conditions, either prescriptive or non-prescriptive formulations, have been mostly developed for the treatments of hemorrhoids and anal fissures, and have been limited to products containing one or two active ingredients comprising pramoxine, decongestants, corticosteroids, analgesics. The method of treatment for the anorectal conditions using the formulations described herein is novel, unique and different from any methods of treatment using any of the existing patented prescriptive and non-prescriptive formulations including those indicated for use in various anorectal conditions such as anal fissures, anal fistulas, anal itch, hemorrhoids, inflammatory bowel diseases, radiation colitis, radiation proctitis, and anorectal infections including bacterial infections, fungal infections, viral infections, and warts/condyomata.

Each of the formulations has a unique combination of several active ingredients as described in the claims, and each of these ingredients exerts its anti-inflammatory effects at various phases of the inflammatory cascade resulting in a synergistic effect of pain relief and healing. The first major active ingredient in the anorectal formulations described herein is selected from a member of the low-, mid-, or high-potency corticosteroids, which are known for their potent anti-inflammatory effects against a wide range of inflammations. Its anti-inflammatory mechanism is mediated through inhibition of prostaglandin synthesis and leukocyte response. Topical corticosteroid preparations, when applied in small quantities, have the advantage of modulating local immune response at the target sites while avoiding systemic side effects and drug interactions. The second major active ingredient in the anorectal formulations described herein is an antidepressant agent(s) with anti-allergic and/or anti-itch properties which can counteract the propagation of the inflammatory cascade through anti-histaminic, serotonin and/or neuronal pathways. The third major active ingredient in the anorectal formulations described herein is a cooling/soothing agent(s), which is crucial in providing immediate relaxation and soothing of the inflamed tissues such as the epithelial, sub-epithelial, mucosal tissues and nerve endings, and in aiding tissue penetration of other active ingredients, thus resulting in an instant relief of symptoms at the target sites. All these ingredients are combined in a commercially accessible cream base, with the overall efficacy resulting from the synergistic effect achieved via combining all or some of the above ingredients are superior to that of the individual ingredient alone.

The novel method of treatment of various anorectal conditions using various novel formulations containing the above three major ingredients are dispersed as an admixture in a commercially accessible cream base formulation.

Taken altogether, the current multi-active ingredient in the anorectal formulations described herein allow each of these ingredients to exist at a lower concentration than the existing single-active ingredient formulations, thus minimizing the likelihood for side effects due to systemic absorption and toxicity. The latter has been the major concern for oral or parenteral administrations of pharmaceutical agents. Such symptoms, in the case of prolonged corticosteroid use, may include reversible hypothalamic-pituitary-adrenal axis suppression, manifestations of Cushing's syndrome, hyperglycemia and glucosuria, and in the case of excessive application of an anti-histaminic/anti-allergic, anti-itch agent(s), may include drowsiness and sedation. On the contrary, systemic absorption leading to clinical symptoms is a rare phenomenon using the formulations as described herein for anorectal administrations, wherein only a small amount of the formulated product is applied on the tip of a finger, pad, swab, cloth or an applicator and then transferred to the target sites comprising the skin, the anus, rectum, anal verge, peri-anal areas, and perineum.

To further enhance its safety margin and efficacy, the anorectal formulations of this invention described herein use lower amounts of each of the multiple active ingredients in lieu of a formulation that contains a larger concentration of a single active ingredient. This is because in the latter preparation, the single active ingredient will have to exist in a much larger quantity in order to achieve the comparable clinical efficacy as the multi-active ingredient formulations. For example, the concern for local untoward side effects such as atrophic changes and telangiectasia in the mucosal/epithelial linings and the anal verge following long-term use of topical steroids at a high concentration can be effectively lessened. The idea leading to the current formulation development parallels the concept of utilizing the multi-drug approach in developing modern chemotherapeutic regimens, i.e., delivering a higher overall efficacy with fewer complications when compared to the single-drug regimens.

The formulations for treatment of anorectal conditions described herein comprise the following selected active ingredients, which are dispersed as an admixture in commercially accessible base formulations. Specifically, the formulations comprise a low-, mid-, or high-potency corticosteroid selected from a listing comprising the following agents/ingredients and/or their salts: alclometasone dipropionate, amcinonide, beclomethasone, betamethasone benzoate, betamethasone dipropionate, betamethasone propionate, betamethasone valerate, budesonide, clobetasol propionate, clobetasone butyrate, clocortolone pivolate, cortisone, desonide, desoximetasone, dexamethasone, diflorasone diacetate, flumethasone pivolate, flunisolide, fluocinolone acetonide, fluocinonide, flurandrenolide, fluticasone propionate, halobetasol propionate, halocinonide, hydrocortisone, hydrocortisone acetate, hydrocortisone butyrate, hydrocortisone probutate, hydrocortisone propionate, hydrocortisone valerate, methylprednisolone, mometasone furoate, prednicarbate, prednisolone, prednisone acetate, prednisone valerate, triamcinolone acetonide, and triamcinolone hexacetonide, with its composition comprising about 0.0005% to about 10% by weight of such an agent(s).

More specifically, the low-mid to mid-potency corticosteroids are selected from a listing comprising the following agents/ingredients and/or their salts: alclometasone dipropionate, clobetasone butyrate, clocortolone pivolate, cortisone, desoximetasone, dexamethasone, hydrocortisone, hydrocortisone acetate, hydrocortisone butyrate, hydrocortisone propionate, hydrocortisone probutate, hydrocortisone valerate, methylprednisolone, prednisolone, prednisone acetate, prednisone valerate, triamcinolone acetonide, and triamcinolone hexacetonide, with its composition comprising about 0.005% to about 10%, preferably about 0.01% to about 5%, and more preferably from about 0.05% to about 2.5% by weight of such an agent(s).

When mid- to high-potency corticosteroids are used, they are selected from a listing comprising the following agents/ingredients and/or their salts: amcinonide, beclomethasone, betamethasone benzoate, betamethasone dipropionate, betamethasone propionate, betamethasone valerate, budesonide, clobetasol propionate, desonide, diflorasone diacetate, flumethasone pivolate, flunisolide, fluocinolone acetonide, fluocinonide, flurandrenolide, fluticasone propionate, halobetasol propionate, halocinonide, mometasone furoate, and prednicarbate, with its composition comprising about 0.0005% to about 5%, preferably about 0.01% to about 3%, and more preferably from about 0.05% to about 1% by weight of such an agent(s).

The formulations for treatment of anorectal conditions also contain an antidepressant agent(s) with anti-allergic and/or anti-itch properties which is selected from a listing comprising the following agents/ingredients and/or their salts: agomelatine, amitryptiline, amoxapine, bupropion, buspirone, carbamazepine, citalopram, clomipramine, desipramine, desvenlafaxine, dexmethylphenidate, dextromethorphan, dosulepin, doxepin, duloxetine, escitalopram, fluoxetine, fluvoxamine, imipramine, isocarboxazid, lamotrigine, lisdexamfetamine, lofepramine, maprotiline, methylphenidate, mianserin, milnacipran, mirtazapine, moclobemide, nefazodone, nortriptyline, opipramol, paroxetine, phenelzine, pirlindole, protriptyline, reboxetine, selegiline, sertraline, sibutramine, tandospirone, tianeptine, tramadol, tranylcypromine, trazodone, trimipramine, valproic acid, venlafaxine, and viloxazine, with its composition comprising about 0.005% to about 20%, preferably about 0.1% to about 10%, and more preferably from about 0.2% to about 5% by weight of such an agent(s).

The formulations for treatment of anorectal conditions also comprise a cooling/soothing agent(s) which is/are selected from a listing comprising the following agents/ingredients and/or their salts/oils: camphor, camphor oil, cinnamon oil, citronella oil, cornmint oil, eucalyptus oil, menthol, methyl salicylate, peppermint oil, and combinations thereof, with its/their composition comprising about 0.001% to about 15%, preferably about 0.1% to about 7.5%, and more preferably from about 0.2% to about 5% by weight of such an agent(s).

2. A method of treatment of and formulations of treatment of genitourinary conditions.

The pathophysiology of symptoms related to various genitourinary conditions shares the final common inflammatory pathway of pain and swelling of the tissues involved, such as the urethral, peri-urethral, vaginal mucosa, labial, vulvar, peri-vaginal, perineal, penile and scrotal epithelia, leading to mucosal and submucosal edema/inflammation, etc. All treatment options aim at relief of tissue swelling and inflammation—the leading cause of pain and discomfort. Current topical preparations for treatments of genitourinary conditions, either prescriptive or non-prescriptive formulations, have been mostly developed for the treatments of post-menopausal atrophic vaginal changes, and often contain one or two active ingredients comprising female sex hormone derivatives, corticosteroids, etc. The method of treatment for the genitourinary conditions using the formulations described herein is novel, unique and different from any methods of treatment using any of the existing patented formulations including prescriptive and non-prescriptive formulations currently indicated for use in various genitourinary conditions such as acute, chronic or recurrent prostatitis, bacterial or non-bacterial prostatitis, prostadynia, balanitis, balanoposthitis, balanitis xerotica obliterans, groin itch, scrotal itch, radiation cystitis, radiation vaginitis, radiation prostatitis, radiation urethritis, infectious and non-infectious urethritis, peri- and post-menopausal urethritis, peri- and post-menopausal vaginitis, atrophic urethritis, urethral syndrome, peri- and post-menopausal vaginitis, infectious and non-infectious vulvovaginitis, atrophic vaginitis, vaginal dryness, non-specific vaginal symptoms, pre- and post-coital discomfort and dyspareunia, vaginismus, vulvodynia, radiation vaginitis, and genitourinary infections including bacterial infections, fungal infections, viral infections, genital herpes, and warts/condyomata.

Each of the formulations for treatment of genitourinary conditions has a unique combination of several active ingredients, and each of these ingredients exerts its anti-inflammatory effects at various phases of the inflammatory cascade resulting in a synergistic effect of pain relief and healing. The first major active ingredient in the current formulations is selected from a member of the low-, mid-, or high-potency corticosteroids, which are known for their potent anti-inflammatory effects against a wide range of inflammations. Its anti-inflammatory mechanism is mediated through inhibition of prostaglandin synthesis and leukocyte response. Topical corticosteroid preparations, when applied in small quantities, have the advantage of modulating local immune response at the target sites while avoiding systemic side effects and drug interactions. The second major active ingredient described herein is an antidepressant agent(s) with anti-allergic and/or anti-itch properties which can counteract the propagation of the inflammatory cascade through anti-histaminic, serotonin and/or neuronal pathways. The third major active ingredient, a cooling or soothing agent(s), is crucial in providing immediate relaxation and soothing of the inflamed tissues such as the epithelial, sub-epithelial, mucosal tissues and nerve endings, and in aiding tissue penetration of other active ingredients, thus resulting in an instant relief of symptoms at the target sites. All these ingredients are combined in a commercially accessible cream base, with the overall efficacy resulting from the synergistic effect achieved via combining all or some of the above ingredients are superior to that of the individual ingredient alone.

The novel method of treatment of various genitourinary conditions using various novel formulations containing the above four major ingredients are dispersed as an admixture in a commercially accessible cream base formulation.

Taken altogether, the current multi-active ingredient formulations allow each of these ingredients to exist at a lower concentration than the existing single-active ingredient formulations, thus minimizing the likelihood for side effects due to systemic absorption and toxicity. The latter has been the major concern for oral or parenteral administrations of pharmaceutical agents. Such symptoms, in the case of prolonged corticosteroid use, may include reversible hypothalamic-pituitary-adrenal axis suppression, manifestations of Cushing's syndrome, hyperglycemia and glucosuria, and in the case of excessive application of an anti-histaminic/anti-allergic, anti-itch agent(s), may include drowsiness and sedation. On the contrary, systemic absorption leading to clinical symptoms is a rare phenomenon using the formulations as described herein for genitourinary administrations, wherein only a small amount of the formulated product is applied on the tip of a finger, pad, swab, cloth or an applicator and then transferred to the target sites comprising the skin, anus, rectum, external genitalia, scrotum, penis, vagina, labia, urethral meatus, peri-urethral area, peri-vaginal area, and perineum.

To further enhance its safety margin and efficacy, these formulations consist of smaller amounts of each of the multiple active ingredients in lieu of a formulation that contains a larger concentration of a single active ingredient. This is because in the latter preparation, the single active ingredient will have to exist in a much larger quantity in order to achieve the comparable clinical efficacy as the multi-active ingredient formulations. For example, the concern for local untoward side effects such as atrophic changes and telangiectasia in the skin or mucosal/epithelial linings following long-term use of topical steroids at a high concentration can be effectively lessened. The idea leading to the current formulation development parallels the concept of utilizing the multi-drug approach in developing modern chemotherapeutic regimens, i.e., delivering a higher overall efficacy with fewer complications when compared to the single-drug regimens.

The formulations for treatment of genitourinary conditions described herein comprise the following selected active ingredients, which are dispersed as an admixture in commercially accessible base formulations. Specifically, the formulations comprise a low-, mid-, or high-potency corticosteroid selected from a listing comprising the following agents/ingredients and/or their salts: alclometasone dipropionate, amcinonide, beclomethasone, betamethasone benzoate, betamethasone dipropionate, betamethasone propionate, betamethasone valerate, budesonide, clobetasol propionate, clobetasone butyrate, clocortolone pivolate, cortisone, desonide, desoximetasone, dexamethasone, diflorasone diacetate, flumethasone pivolate, flunisolide, fluocinolone acetonide, fluocinonide, flurandrenolide, fluticasone propionate, halobetasol propionate, halocinonide, hydrocortisone, hydrocortisone acetate, hydrocortisone butyrate, hydrocortisone probutate, hydrocortisone propionate, hydrocortisone valerate, methylprednisolone, mometasone furoate, prednicarbate, prednisolone, prednisone acetate, prednisone valerate, triamcinolone acetonide, and triamcinolone hexacetonide, with its composition comprising about 0.0005% to about 10% by weight of such an agent(s).

More specifically, the low-mid to mid-potency corticosteroids are selected from a listing comprising the following agents/ingredients and/or their salts: alclometasone dipropionate, clobetasone butyrate, clocortolone pivolate, cortisone, desoximetasone, dexamethasone, hydrocortisone, hydrocortisone acetate, hydrocortisone butyrate, hydrocortisone propionate, hydrocortisone probutate, hydrocortisone valerate, methylprednisolone, prednisolone, prednisone acetate, prednisone valerate, triamcinolone acetonide, and triamcinolone hexacetonide, with its composition comprising about 0.005% to about 10%, preferably about 0.01% to about 5%, and more preferably from about 0.05% to about 2.5% by weight of such an agent(s).

When mid- to high-potency corticosteroids are used, they are selected from a listing comprising the following agents/ingredients and/or their salts: amcinonide, beclomethasone, betamethasone benzoate, betamethasone dipropionate, betamethasone propionate, betamethasone valerate, budesonide, clobetasol propionate, desonide, diflorasone diacetate, flumethasone pivolate, flunisolide, fluocinolone acetonide, fluocinonide, flurandrenolide, fluticasone propionate, halobetasol propionate, halocinonide, mometasone furoate, and prednicarbate, with its composition comprising about 0.0005% to about 5%, preferably about 0.01% to about 3%, and more preferably from about 0.05% to about 1% by weight of such an agent(s).

The formulations for treatment of genitourinary conditions also contain an antidepressant agent(s) with anti-allergic and/or anti-itch properties which is selected from a listing comprising the following agents/ingredients and/or their salts: agomelatine, amitryptiline, amoxapine, bupropion, buspirone, carbamazepine, citalopram, clomipramine, desipramine, desvenlafaxine, dexmethylphenidate, dextromethorphan, dosulepin, doxepin, duloxetine, escitalopram, fluoxetine, fluvoxamine, imipramine, isocarboxazid, lamotrigine, lisdexamfetamine, lofepramine, maprotiline, methylphenidate, mianserin, milnacipran, mirtazapine, moclobemide, nefazodone, nortriptyline, opipramol, paroxetine, phenelzine, pirlindole, protriptyline, reboxetine, selegiline, sertraline, sibutramine, tandospirone, tianeptine, tramadol, tranylcypromine, trazodone, trimipramine, valproic acid, venlafaxine, and viloxazine, with its composition comprising about 0.005% to about 20%, preferably about 0.1% to about 10%, and more preferably from about 0.2% to about 5% by weight of such an agent(s).

The formulations for treatment of genitourinary conditions also comprise a cooling/soothing agent(s) which is/are selected from a listing comprising the following agents/ingredients and/or their salts/oils: camphor, camphor oil, cinnamon oil, citronella oil, cornmint oil, eucalyptus oil, menthol, methyl salicylate, peppermint oil, and combinations thereof, with its/their composition comprising about 0.001% to about 15%, preferably about 0.1% to about 7.5%, and more preferably from about 0.2% to about 5% by weight of such an agent(s).

It should be emphasized that the above-described embodiments of the present disclosure are merely possible examples of implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and the present disclosure and protected by the following claims.

While the foregoing is directed to embodiments of the present invention, other and further embodiments of the invention may be devised without departing from the basic scope thereof. For example, although numerous embodiments having various features have been described herein, combinations of such various features in other combinations not discussed herein are contemplated within the scope of embodiments of the present invention.

What is claimed is:

1. A topically-administered composition for treating inflammation-related medical conditions, the composition consisting of active ingredients and inactive ingredients, the active ingredients consisting of:
(a) a corticosteroid ingredient, or salt thereof, selected from the group consisting of alclometasone dipropionate, amcinonide, beclomethasone, betamethasone benzoate, betamethasone dipropionate, betamethasone propionate, betamethasone valerate, budesonide, clobetasol propionate, clobetasone butyrate, clocortolone pivolate, cortisone, desonide, desoximetasone, dexamethasone, diflorasone diacetate, flumethasone pivolate, flunisolide, fluocinolone acetonide, fluocinonide, flurandrenolide, fluticasone propionate, halobetasol propionate, halocinonide, hydrocortisone, hydrocortisone acetate, hydrocortisone butyrate, hydrocortisone probutate, hydrocortisone propionate, hydrocortisone valerate, methylprednisolone, mometasone furoate, prednicarbate, prednisolone, prednisone acetate, prednisone valerate, triamcinolone acetonide, and triamcinolone hexacetonide;
(b) an anti-depressant agent(s) ingredient, or salt thereof, selected from the group consisting of agomelatine, amitryptiline, amoxapine, bupropion, buspirone, carbamazepine, citalopram, clomipramine, desipramine, desvenlafaxine, dexmethylphenidate, dextromethorphan, dosulepin, doxepin, duloxetine, escitalopram, fluoxetine, fluvoxamine, imipramine, isocarboxazid, lamotrigine, lisdexamfetamine, lofepramine, maprotiline, methylphenidate, mianserin, milnacipran, mirtazapine, moclobemide, nefazodone, nortriptyline, opipramol, paroxetine, phenelzine, pirlindole, protriptyline, reboxetine, selegiline, sertraline, sibutramine, tandospirone, tianeptine, tramadol, tranylcypromine, trazodone, trimipramine, valproic acid, venlafaxine, and viloxazine;
(c) a cooling/soothing agent(s) ingredient(s), or salt(s) or oil(s) thereof, selected from the group consisting of camphor, camphor oil, cinnamon oil, citronella oil, cornmint oil, eucalyptus oil, menthol, methyl salicylate, and peppermint oil, and combinations thereof;
wherein said corticosteroid agent(s) is present in an amount of from about 0.0005% to about 10% by weight, said anti-depressant agent(s) is present in an amount of from about 0.005% to about 20% by weight, and said cooling/soothing agent(s) are present in an amount of from about 0.001% to about 15% by weight; and
wherein the inactive ingredients consist of a commercially accessible cream base.

2. The composition according to claim 1 wherein said corticosteroid agent(s) is a low-mid to mid-potency corticosteroid, or salt thereof, selected from the group consisting of alclometasone dipropionate, clobetasone butyrate, clocortolone pivolate, cortisone, desoximetasone, dexamethasone, hydrocortisone, hydrocortisone acetate, hydrocortisone butyrate, hydrocortisone propionate, hydrocortisone probutate, hydrocortisone valerate, methylprednisolone, prednisolone, prednisone acetate, prednisone valerate, triamcinolone acetonide, and triamcinolone hexacetonide.

3. The composition according to claim 1 wherein said corticosteroid is a mid- to high-potency corticosteroid, or salt thereof, selected from the group consisting of amcinonide, beclomethasone, betamethasone benzoate, betamethasone dipropionate, betamethasone propionate, betamethasone valerate, budesonide, clobetasol propionate, desonide, diflorasone diacetate, flumethasone pivolate, flunisolide, fluocinolone acetonide, fluocinonide, flurandrenolide, fluticasone propionate, halobetasol propionate, halocinonide, mometasone furoate, and prednicarbate.

4. A method of treatment of inflammation-related anorectal medical conditions by delivering a composition according to claim 1, 2 or 3 comprising the steps of:
(a) diagnosing one or more of said anorectal conditions in a patient;
(b) formulating said composition by dispersing the four major active ingredients into a commercially accessible cream base; and
(c) delivering an effective amount of said formulation topically to the affected areas of a patient in need thereof by way of bare finger, gloved finger, pad, swab, cloth, applicator, or patch;
wherein said anorectal conditions comprise anal fissures, anal fistulas, anal itch, hemorrhoids, inflammatory bowel diseases, radiation colitis, radiation proctitis, and anorectal infections including bacterial infections, fungal infections, viral infections, and warts/condyomata, and said affected areas comprise the skin, the anus, rectum, anal verge, peri-anal areas, and perineum, and said patient is a human.

5. A method of treatment of inflammation-related genitourinary medical conditions by delivering a composition according to claim 1, 2 or 3 comprising the steps of:
(a) diagnosing one or more of said genitourinary conditions in a patient;
(b) formulating said composition by dispersing the three major active ingredients into a commercially accessible base; and
(c) delivering an effective amount of said formulation topically to the affected areas of a patient in need thereof by way of bare finger, gloved finger, pad, swab, cloth, applicator, or patch;
wherein said genitourinary conditions comprise acute, chronic or recurrent prostatitis, bacterial or non-bacterial prostatitis, prostadynia, balanitis, balanoposthitis, balanitis xerotica obliterans, groin itch, scrotal itch, radiation cystitis, radiation vaginitis, radiation prostatitis, radiation urethritis, infectious and non-infectious urethritis, peri- and post-menopausal urethritis, peri- and post-menopausal vaginitis, atrophic urethritis, urethral syndrome, peri- and post-menopausal vaginitis, infectious and non-infectious vulvovaginitis, atrophic vaginitis, vaginal dryness, non-specific vaginal symptoms, pre- and post-coital discomfort and dyspareunia, vaginismus, vulvodynia, radiation vaginitis, and genitourinary infections including bacterial infections, fungal infections, viral infections, genital herpes, and warts/condyomata, and said affected areas comprise the skin, anus, rectum, external genitalia, scrotum, penis, vagina, labia, urethral meatus, peri-urethral area, peri-vaginal area, and perineum, and said patient is a human.

6. The composition according to claims 1, 4 and 5 wherein said commercially accessible cream base may contain ingredients selected from the group consisting of mineral oil, petrolatum, paraffin, white wax, emulsifying wax, alcohol, glycerin, glycerol, glyceryl stearate, glyceryl triacetate, glycol, glycol stearate, alkyl benzoate, benzoic acid, benzyl alcohol, ceresin, cetearyl alcohol, cetostearyl alcohol, cetyl alcohol, cetyl ester, sodium cetostearyl sulphate, isopropyl myristate, isopropyl palmitate, lactic acid, lanolin, lanolin alcohol, methylchloroisothiazolinone, methylisothiazolinone, polydextrose, sorbic acid, sorbitan, stearate, stearic acid, stearyl alcohol, polysorbate, sodium hydroxide, parabens, phenoxyethanol, triethanolamine, sodium sulphite, sodium metabisulfite, propylene glycol, polyethylene glycol stearate, chlorocresol, sodium citrate, citric acid, sorbitol, maltitol, titanium dioxide, glyceryl polymethacrylate, polyethylene glycol glyceryl stearate, dimethiconol, dimethicone, ether, polyoxy stearate, polyethylene fatty acid ester, and combinations thereof, with its/their composition comprising about 0.001% to about 35% by weight of such an agent(s) and/or its salt(s), and with water comprising about 5% to about 65% by weight.
